(19) United States
(12) Patent Application Publication
(10) Pub. No.: US 2007/0111267 A1
(43) Pub. Date: May 17, 2007

(54) Method of determining efficacy of anticancer drug and determination kit therefor

(76) Inventors: Jun-ya Kato, Kyoto (JP); Kiichiro Tomoda, Osaka (JP); Noriko Kato, Kyoto (JP)

Correspondence Address: SHERIDAN ROSS PC, 1560 BROADWAY, SUITE 1200, DENVER, CO, 80202, US

(21) Appl. No.: 10/558,218

(22) PCT Filed: Nov. 20, 2003
(86) PCT No.: PCT/JP04/14853

§ 371 Date: Sep. 13, 2006

(30) Foreign Application Priority Data
May 23, 2003 (JP) 2003-146899

(51) Int. Cl. G01N 33/574

(52) U.S. Cl. 435/7.23

(57) ABSTRACT

A method of determining the efficacy of anticancer drug whereby accurate determination can be realized; and a determination kit therefor. In the determination of the efficacy of anticancer drugs, especially a tyrosine kinase inhibitor, Jab1 protein is used as an index of determination and the form of existence of Jab1 protein is checked. As the form of existence there can be mentioned Jab1 small complex of 120 kDa molecular weight, and the type having sensitivity to the efficacy of anticancer drug can be accurately determined by checking the expression of p27 protein.

### TECHNICAL FIELD

The present invention relates to a method of determining the efficacy of anticancer drugs, whereby the effectiveness of anticancer drug administered to cancer patients is checked, and to a determination kit therefor. The invention particularly relates to a determination method suitable for checking the effectiveness of a tyrosine kinase inhibitor used in the treatment of, for example, Chronic Myelogenous Leukemia (CML), and to a determination kit used to implement the method.

### BACKGROUND ART

A large proportion of cancer treatment involves chemotherapy using anticancer drugs. However, the problem of chemotherapy using anticancer drugs is that it is difficult to predict the efficacy of the anticancer drug, and that the patients must go through mental and physical distresses caused by the side effects of the treatment. It is therefore important to predict the effectiveness of a specific anticancer drug to a particular cancer patient. That is, predicting the efficacy of anticancer drugs is highly important in chemotherapy.

For example, in Chronic Myelogenous Leukemia (hereinafter, simply “CML” where appropriate), a tyrosine kinase inhibitor has been developed as a specific anticancer drug. The tyrosine kinase inhibitor targets gene abnormality in CML cancer cells with minimal effect on normal cells. A representative example of the tyrosine kinase inhibitor is Gleevec™/Glivec® (Novartis).

The following will describe the action of the tyrosine kinase inhibitor in more detail. Bcr-Abl gene is known as a causal factor of CML, and it encodes P210 protein. The tyrosine kinase inhibitor inhibits the tyrosine kinase activity of the P210 protein and thereby induces apotosis, with the result that abnormal proliferation of leucocytes is suppressed.

As such, in using the tyrosine kinase inhibitor as an anticancer drug, whether or not the tyrosine kinase inhibitor will be effective for the CML patients, i.e., the efficacy of the tyrosine kinase inhibitor, has been determined based on existence and expression of the Bcr-Abl gene (see Manley P W et al. Eur J. Cancer. 2002 September; 38 Suppl 5: S19-27, for example).

Meanwhile, there have been reports recently concerning Bcr-Abl mutant gene that has been mutated to be resistant to the tyrosine kinase inhibitor, and examples of cancer cells, resulting from multiple mutations, that do not depend on the activity of the P210 protein encoded by Bcr-Abl gene (see Hochhaus A et al. Leukemia. 2002 November; 16 (11): 2190-6, for example). This is considered to be the result of the nature of cancer cells, which tend to accumulate genetic mutations.

Further, the inventors of the present invention have reported that Jab1 protein specifically binds to p27 protein, and that Jab1 protein is associated with localized degradation of p27 protein in the cell (see Tomoda K et al. Nature. 1999 Mar. 11; 398 (6723): 160-5, and Tomoda K et al. J Biol. Chem. 2002 Jan. 18; 277 (3): 2302-10, for example).

In this connection, the inventors of the present invention have also reported that the expression of Jab1 is controlled by Bcr-Abl, and that subsequent induction of p27 degradation may be responsible for the development of cancer (see the program and lecture summary for the 25th Annual Meeting of the Molecular Biology Society of Japan, 2P-0760, organizing committee of the 25th Annual Meeting of the Molecular Biology Society of Japan, published on Nov. 25, 2002).

The existence of Bcr-Abl mutant gene or the existence of cancer cells that do not depend on the activity of P210 protein brings uncertainty to the conventional determination method in which Bcr-Abl gene is used as an index.

Thus, the conventional determination method may erroneously yield a positive test result for a patient negative to the tyrosine kinase inhibitor. In this case, the tyrosine kinase inhibitor is unnecessarily administered to the patient while there is no efficacy in doing so. As a result, the effectiveness of the anticancer drug is reduced, and the patient must endure the side effects and huge cost of the anticancer drug.

The present invention was made in view of the foregoing problems, and it is an object of the present invention to provide a method of determining the efficacy of anticancer drugs, whereby the effectiveness of the anticancer drug, tyrosine kinase inhibitor, can be accurately checked. The invention also provides a determination kit used therefor.

### DISCLOSURE OF INVENTION

The inventors of the present invention diligently worked to solve the foregoing problems, and accomplished the invention by finding that Jab1 protein, which is a molecule that specifically binds to Cdk inhibitor p27 protein, exists in various forms, and that one form of Jab1 protein has efficacy corresponding to that of a tyrosine kinase inhibitor.

According to the present invention, there is provided a method of determining the efficacy of anticancer drug. Specifically, in order to solve the foregoing problems, the present invention provides a method of determining the efficacy of an anticancer drug used for cancer treatment, wherein Jab1 protein contained in a test sample is used as an index of determination.

The method of determining the efficacy of anticancer drug preferably includes an extract preparing step of preparing an extract from a test sample containing cancer cells; and a Jab1 checking step of checking for a form of existence of Jab1 protein contained in the extract. More preferably, the method includes a fractionating step of fractionating the extract based on molecular weight, wherein the extract fractionated in the fractionating step is used in the Jab1 checking step.

In the Jab1 checking step, it is preferable that a molecular weight of a Jab1 complex formed by the Jab1 protein be used as a reference to check for a form of existence of Jab1 protein. Here, the Jab1 complex exists as a Jab1 large complex with a molecular weight of 450 kDa, and/or a Jab1 small complex with a molecular weight of 120 kDa, and the Jab1 small complex has sensitivity to the efficacy of the anticancer drug. It is therefore preferable that, in the Jab1 checking step, an expression level of the Jab1 small complex be checked to check for a form of existence of Jab1 protein.

Further, a method of determining the efficacy of anticancer drug according to the present invention preferably includes a p27 checking step of checking for expression of p27 protein contained in the test sample. This is because the Jab1 small complex exists in different sub types, including those sensitive to the efficacy of anticancer drug, and those not sensitive to the efficacy of anticancer drug. By checking the expression of p27 protein, a type of Jab1 small complex having sensitivity to the efficacy of anticancer drug can be determined more accurately.

In at least one of the Jab1 checking step and the p27 checking step, an expression level of the Jab1 small complex or protein is checked by electrophoresis. In the Jab1 checking step, it is preferable that the electrophoresis be performed by native-polyacrylamide gel electrophoresis. This is to prevent the Jab1 small complex from being dissociated due to denaturation, and thereby make sure that the Jab1 small complex is checked.

In the Jab1 checking step, it is preferable that the Jab1 small complex be detected from the extract using an antibody that specifically binds to the Jab1 protein. Similarly, in the p27 checking step, it is preferable that the p27 protein be detected from the extract using an antibody that specifically binds to the p27 protein. Here, the antibody is either one of a monoclonal antibody and a polyclonal antibody.

The type of cancer to which the method of determining the efficacy of anticancer drug according to the present invention is applicable is not particularly limited. However, the cancer is preferably leukemia, and more preferably Chronic Myelogenous Leukemia. The type of anticancer drug to which the method of determining the efficacy of anticancer drug according to the present invention is applicable is not particularly limited. However, a tyrosine kinase inhibitor is preferable. It is highly preferably that the method of determining the efficacy of anticancer drug according to the present invention be performed prior to administration of the anticancer drug.

An anticancer drug efficacy determination kit according to the present invention is used to perform the foregoing method of determining the efficacy of anticancer drug. In one specific example, the anticancer drug efficacy determination kit includes a group of reagents for checking a state of expression of Jab1 protein contained in the extract prepared from a test sample including cancer cells. It is preferable that the group of reagents include an antibody that specifically binds to Jab1 protein. It is also preferable that the group of reagents include at least Jab1 small complex of the Jab1 complex as a marker for polyacrylamide gel electrophoresis.

According to the foregoing configuration, a form of existence of Jab1 protein is checked by using the Jab1 protein as an index of determination, thereby evaluating the sensitivity of cancer to the anticancer drug. Thus, the efficacy of the anticancer drug can be accurately determined prior to administration of the anticancer drug.

For a fuller understanding of the nature and advantages of the invention, reference should be made to the ensuing detailed description taken in conjunction with the accompanying drawings.

### BRIEF DESCRIPTION OF DRAWINGS

FIG. 1(A) is a view illustrating results of Western blotting detecting the expression level of proteins contained in various leukemia cell lines; FIG. 1(B) is a view illustrating results of Northern blotting detecting the expression level of mRNA of the proteins contained in various leukemia cells; FIG. 1(C) is a view illustrating results of Western blotting detecting Jab1 protein contained in each fraction of cell extracts obtained from K562 and KYO-1; and FIG. 1(D) is a view illustrating results of Western blotting detecting Jab1 complex contained in the K562 cell extract and its fraction.

FIG. 2(A) is a view illustrating results of Western blotting performed on the cell extracts using an anti-Jab1 antibody; and FIG. 2(B) is a view illustrating results of Western blotting performed on Jab1 protein and other proteins contained in the cell extracts obtained from 5 kinds of CML cell lines and 2 kinds of non-CML cells.

FIG. 3(A) is a view illustrating results of analysis on changes in Jab1 complex, the level of phosphorylated tyrosine, and the expression level of Jab1 protein separated by Native-PAGE performed on various types of cell extracts that have been treated and have not been treated with tyrosine kinase inhibitor; and FIG. 3(B) is a view illustrating results of Western blotting analyzing the expression levels of p27 protein, Cul1 protein, Skp2 protein, Bcr-Abl protein, cAbl protein, and γ-Tub protein separated by SDS-PAGE performed on various types of cell extracts.

FIG. 4 is a view illustrating results of analysis on the relationship between Bcr-Abl gene induction and Jab1 complex, using mouse pro-B cell line BAF3 and TonB cells.

FIG. 5(A) is a view illustrating results of Western blotting analyzing changes in Jab1 complex and the expression level of different proteins in the cell extracts obtained from K562 treated with various chemicals; FIG. 5(B) is a view illustrating results of Western blotting analyzing the expression level of p27 protein in the cell extracts obtained from KYO-1 treated with various chemicals; and FIG. 5(C) is a view illustrating results of Western blotting analyzing changes in Jab1 complex in the cell extracts obtained from K572 treated with different combinations of chemicals.

### BEST MODE FOR CARRYING OUT THE INVENTION

The following will describe the present invention in more detail.

One embodiment of the present invention is described below. It should be noted however that the invention is not limited in any way by the following description.

In order to determine the efficacy (drug efficacy) of anticancer drug for Chronic Myelogenous Leukemia (CML) prior to administration of the drug, Jab1 protein contained in a test sample obtained from cancer patients is used as an index in the present invention. In this way, the efficacy of the anticancer drug can be determined and predicted prior to use, thereby suitably selecting an anticancer drug for each different patient.

In the following, description is made as to a specific example of a determination method according to the present invention, an example of a determination kit used therefor, and use of the present invention.

(I) Method of Determining the Efficacy of Anticancer Drugs

A determination method according to the present invention determines the efficacy of anticancer drugs used for cancer treatment, wherein Jab1 protein contained in a test sample is used as an index of determination. A specific technique by which Jab1 protein is used as an index of determination is not particularly limited as long as it enables the state of expression of the Jab1 protein to be checked.

<Jab1 Protein and Jab1 Complex>

Jab1 protein has been identified as a factor that specifically binds to p27 protein, which is a cyclin-dependent kinase (Cdk, an enzyme involved in phosphorylation under the control of cyclin, which is a protein acting to control the cell cycle) inhibitor, i.e., a factor associated with localized degradation of p27 protein in the cell. Cancer cells are best characterized by abnormal proliferation of cells. A disrupted cell cycle, and particularly disrupted G1 control mechanism is one of the factors of cancer development. The p27 protein is a molecule that negatively controls the progress of the G1 period. In various types of cancer, there have been reports suggesting a negative correlation between a reduced expression level of p27 protein and malignancy of cancer.

In this connection, the inventors of the present invention studied the state of expression of Jab1 protein in a variety of leukemia cells, and found that the Jab1 protein formed a complex with other factors, and that the complex (Jab1 complex) was a COP signalosome complex (Jab1 large complex) with a molecular weight of 450 kDa, and/or a Jab1 small complex with a molecular weight of 120 kDa.

While the Jab1 large complex was commonly found in all cells, the Jab1 small complex was found only in cancer cells expressing the Bcr-Abl gene. The Bcr-Abl gene is a chimera gene generated by the translocation between chromosome 9 and chromosome 22, and it resides on the Philadelphia chromosome, which is present in more than 90% of CML patients. Abl gene on chromosome 9 codes for a non-receptor tyrosine kinase. The protein encoded by cancerous v-abl gene has strong kinase activity, whereas the protein encoded by normal abl gene hardly shows tyrosine kinase activity. Bcr gene resides on chromosome 22, and it codes for serine kinase. The Bcr-Anl gene, which is the fusion of these genes, is known to code for a protein (P210 protein) that exhibits strong serine and tyrosine kinase activity.

It was also found that inhibiting the tyrosine kinase activity of P210 protein encoded by Bcr-Abl gene reduces the amount of Jab1 small complex, while the amount of Jab1 large complex remained unchanged. Inversely, inducing the expression of Bcr-Abl gene increased the amount of Jab1 small complex, while the amount of Jab1 large complex remained unchanged. It was also revealed that the Jab1 small complex exists in at least two forms with different mobilities in electrophoresis. (See the program and lecture summary for the 25th Annual Meeting of the Molecular Biology Society of Japan, 2P-0760, organizing committee of the 25th Annual Meeting of the Molecular Biology Society of Japan, published on Nov. 25, 2002. See also Example 1 below.)

From these results, it was found that the Jab1 small complex of the Jab1 complex was controlled by Bcr-Abl gene.

Further, using the tyrosine kinase inhibitor as an anticancer drug, the inventors of the present invention studied relationships between the two kinds of Jab1 small complexes and the efficacy of the anticancer drug. It was found as a result that the treatment with the tyrosine kinase inhibitor reduces the amount of one of the Jab1 small complexes and increases the amount of p27 protein. As for the other Jab1 small complex, the amount of Jab1 small complex was reduced upon treatment with the tyrosine kinase inhibitor, but the amount of p27 protein did not change in this case. (See the program and lecture summary for the 25th Annual Meeting of the Molecular Biology Society of Japan, 2P-0760, organizing committee of the 25th Annual Meeting of the Molecular Biology Society of Japan, published on Nov. 25, 2002. See also Example 2 below.)

From these results, it was found that the different forms of Jab1 small complex are one of the factors controlling the expression of p27 protein, and that these different forms are therefore involved in sensitivity to the tyrosine kinase inhibitor.

In sum, the inventors of the present invention found that Jab1 protein acts downstream of Bcr-Abl gene and upstream of p27 gene, and that the Jab1 protein exists in different forms, with one of the forms corresponding to the efficacy of the tyrosine kinase inhibitor.

Thus, in using an anticancer drug and particularly the tyrosine kinase inhibitor, the efficacy of the anticancer drug can be determined by observing forms of existence of Jab1 protein included in a test sample obtained from a cancer patient, before the anticancer drug is administered to the cancer patient.

<Example of Determination Method>

A method of determining the efficacy of anticancer drugs according to the present invention is not particularly limited as long as it checks for forms of existence of Jab1 protein included in a test sample, as described above. In one specific example, the method includes an extract preparing step, a fractionating step, a Jab1 checking step, and a p27 checking step.

A test sample used in the present invention is not particularly limited as long as it includes, or presumably includes, cancer cells. In the present invention, a tyrosine kinase inhibitor specific to CML is used as an anticancer drug, and therefore the test sample preferably includes leucocytes obtained from, for example, peripheral blood or bone marrow fluid. More specifically, the test sample may include one or more of the following: leucocytes, for example, such as lymphocytes (T cells, B cells), granulocytes (neutrophils, eosinophiles, basophils), monocytes, macrophage, mast cells, and natural killer cells; haematopoietic stem cells; and lymphocytic stem cells.

A test sample used in the present invention may be one directly obtained from humans by collecting, for example, blood, bone marrow fluid, or a body fluid containing leucocytes. Alternatively, the collected blood or body fluid may be subjected to conventional treatments and processed into form that allows molecular biological analyses to be performed more easily.

The extract preparing step is not particularly limited as long as an extract is prepared from the test sample. Specifically, for example, separated cells from the test sample are disrupted and then dissolved or suspended in a suitable buffer solution. The method of disrupting the cells is not particularly limited, and conventional methods such as an ultrasonic disruption method can be used.

The fractionating step is not particularly limited as long as the extract obtained in the extract preparing step is fractionated based on molecular weight. Specific examples include electrophoresis methods such as native polyacrylamide gel electrophoresis (Native-PAGE), SDS-polyacrylamide gel electrophoresis (SDS-PAGE), and agarose gel electrophoresis; density gradient methods using glycerol or the like; methods using an ultrafiltration membrane; and gel filtration. The conditions under which these methods are performed, and the method of collecting fractions are not particularly limited either.

The Jab1 checking step is not particularly limited as long as forms of existence of Jab1 protein contained in the extract are checked for. In the present invention, it is preferable that the extract fractionated in the fractionating step be used in the Jab1 checking step. This is preferable because it allows the forms of existence of Jab1 protein to be checked more accurately. It should be noted however that a crude extract may be used instead of the extract obtained in the fractionating step, if it allows forms of existence of Jab1 protein to be sufficiently checked in the Jab1 checking step.

In one specific technique of checking forms of existence of Jab1 protein in the Jab1 checking step, the molecular weight of Jab1 complex formed by Jab1 protein is used as a reference. As described above, the Jab1 complex exists as a Jab1 large complex with a molecular weight of 450 kDa, and/or a Jab1 small complex with a molecular weight of 120 kDa. Since the Jab1 small complex is controlled by Bcr-Abl gene, forms of existence of Jab1 protein can be checked in the Jab1 checking step by checking the expression level of Jab1 small complex.

The Jab1 small complex exists in at least two sub types. In one type of Jab1 small complex having sensitivity to the tyrosine kinase inhibitor, treatment with the tyrosine kinase inhibitor reduces the amount of Jab1 small complex, and increases the amount of p27 protein. It is therefore preferable that a determination method according to the present invention include, in addition to the Jab1 checking step, a p27 checking step of checking expression of p27 protein contained in the test sample.

Specific methods by which the Jab1 checking step and the p27 checking step are performed are not particularly limited, and conventional methods can be used therefor. Generally, a technique of checking the expression level by electrophoresis is preferably used. The electrophoresis may be any of the foregoing electrophoresis methods described in conjunction with the fractionating step. Among these methods, the Native-PAGE method is particularly preferable in the Jab1 checking step.

As described above, the Jab1 protein checked in the Jab1 checking step forms a complex. Thus, with the technique in which denatured protein is electrophorased as in SDS-PAGE, it is difficult to sufficiently check for the presence of Jab1 complex (Jab1 small complex). It is therefore highly preferable that the present invention use the Native-PAGE method particularly in the Jab1 checking step.

The method of detecting the Jab1 small complex in the Jab1 checking step is not particularly limited. Generally, an antibody (anti-Jab1 antibody) that specifically binds to Jab1 protein is used. That is, the detection method is not limited to a specific method and conventional methods can be used. In one preferable method, conventional Western blotting is employed using an enzyme-labeled or radioisotope-labeled anti-Jab1 antibody. In the case where the extract used in the Jab1 checking step is a sample fractionated by a density gradient method, detection can be made by a conventional dot blotting method.

The method of detecting p27 protein is not particularly limited, and, as in the Jab1 checking step, an antibody (anti-p27 antibody) that specifically binds to p27 protein can be used. That is, as in the Jab1 checking step, the detection method is not limited to a specific method, and conventional Western blotting methods can be used.

The anti-Jab1 antibody used in the Jab1 checking step, and the anti-p27 antibody used in the p27 checking step are not particularly limited as long as they can recognize an antigen determining group formed by at least a partial structure of the antigen (Jab1 protein, p27 protein), and immunologically detect these proteins without fail. Specifically, the antibody may be a polyclonal antibody or a monoclonal antibody, of which the latter is more preferable since it has higher specificity.

The anti-Jab1 antibody and anti-p27 antibody may be produced by conventional methods, or may be commercially available products. The method of producing the antibody is not particularly limited. For example, in the case of monoclonal antibody, the antibody may be produced from hybridoma that has been prepared by fusing splenic lymphocytes and bone marrow cells of a mouse immunized with Jab1 protein or p27 protein. In the case of polyclonal antibody, the antibody may be purified from the immune serum of a rabbit immunized with Jab1 protein or p27 protein.

The timing of performing a determination method according to the present invention is not particularly limited. In principle, it is highly preferable that the method be performed prior to administration of the anticancer drug to cancer patients. By determining the efficacy of the anticancer drug before administration, effectiveness of the anticancer drug can be improved. Further, patients do not need to endure the side effects or huge cost of the anticancer drug which would not have been needed.

The determination method described in this embodiment may include other processes and steps. Further, the order of the steps described above may be changed as required.

As described above, in a determination method according to the present invention, Jab1 protein is used as an index of determination, and forms of existence of Jab1 protein are checked in particular, thereby evaluating sensitivity of cancer to the tyrosine kinase inhibitor. In this way, the efficacy of the tyrosine kinase inhibitor, a specific anticancer drug, can be accurately determined prior to administration of the anticancer drug.

(II) Anti-Cancer Drug Efficacy Determination Kit

An anticancer drug efficacy determination kit according to the present invention is used to perform the foregoing method of determining the efficacy of anticancer drugs. The anticancer drug efficacy determination kit is not limited to a particular structure as long as it includes reagents and materials least required for performing the method. Specifically, the anticancer drug efficacy determination kit may include a group of reagents for checking a state of expression of Jab1 protein, for example.

Such a group of reagents is not particularly limited, and may include, for example, a buffer solution of a composition suitable for preparing an extract from a test sample containing cancer cells, the anti-Jab1 antibody used in the Jab1 checking step, the anti-p27 antibody used in the p27 checking step, and fractionating reagents (for example, glycerol used in a density gradient method) used in the fractionating step.

In a determination method according to the present invention, it is preferable that Native-PAGE and Western blotting be successively carried out in the Jab1 checking step and the p27 checking step. It is therefore preferable that a determination kit according to the present invention include proteins that serve as markers in Native-PAGE, and the anti-Jab1 antibody and/or anti-p27 antibody.

Preferably, the marker proteins include at least the Jab1 small complex with a molecular weight of 120 kDa, and more preferably the Jab1 large complex with a molecular weight of 450 kDa. It is also preferable to include p27 protein. These proteins should be purified to such an extent that their molecular weights can be clearly identified by PAGE. Further, these proteins may be used as independent markers or mixed markers.

Further, a determination kit according to the present invention may include elements other than the group of reagents set forth above. For example, the kit may include a material such as a micro tube of a size suitable for dispensing the extract. Further, a determination kit according to the present invention may be used in combination with an anticancer drug applicable to the present invention. In other words, a determination kit according to the present invention may include an anticancer drug such as the tyrosine kinase inhibitor. In this case, the determination kit can be more appropriately referred to as an anticancer drug kit.

With a determination method of the present invention provided as a kit, the efficacy of anticancer drugs to cancer patients can be determined more easily and more accurately. The present invention is therefore usable in industrial settings such as laboratory testing and pharmaceutical industry.

(III) Use of the Present Invention

The inventors of the present invention studied pathways of intracellular signal transduction associated with Bcr-Abl gene, using, as an index, the expression of p27 (Cdk inhibitor) protein, which is a negative marker for malignant cancer. It was found as a result that Jab1 protein acts downstream of Bcr-Abl gene and upstream of p27 gene, and that the Jab1 protein exists in different forms (Jab1 complex), with one of the forms corresponding to the efficacy of the specific anticancer drug, tyrosine kinase inhibitor. Thus, the determination method and/or determination kit according to the present invention can be used in a wide variety of area where chemotherapy is employed to treat cancer associated with Bcr-Abl gene.

Specifically, the type of cancer to which the present invention is applicable is not particularly limited as long as it has active tyrosine kinase. Specific examples of such cancer include: leukemia, vesicular lung cancer, prostatic cancer, breast cancer, pancreatic cancer, soft part sarcoma, and human gastrointestinal stroma (GIST), which is one type of gastrointestinal solid carcinoma. Of these, leukemia, and CML in particular, are preferable.

The anticancer drug to which the present invention is applicable is not particularly limited as long as it is a tyrosine kinase inhibitor. A representative example of a tyrosine kinase inhibitor is the Chronic Myelogenous Leukemia treatment drug developed and put to actual practice by the Novartis of the Switzerland and sold under the commercial name Gleevec™/Glivec® (STI571). Gleevec™/Glivec® is a compound called imatinib mesylate, and it is designed such that the molecule selectively binds to the ATP binding site of P210 protein encoded by Bcr-Abl gene of the CML patient. Though detailed mechanisms of action are unknown, it is believed that the compound inhibits tyrosine kinase activity of P210 protein and thereby suppresses abnormal proliferation of leucocytes by induced apotosis. With the present invention, the anticancer efficacy of Gleevec™/Glivec® can be accurately determined. The present invention is also applicable to other products such as IRESSA® (the product of AstroZeneca), and Herceptin (the product of Nippon Roche).

The present invention is not limited to the description of the embodiments above, but may be altered by a skilled person within the scope of the claims. An embodiment based on a proper combination of technical means disclosed in different embodiments is encompassed in the technical scope of the present invention.

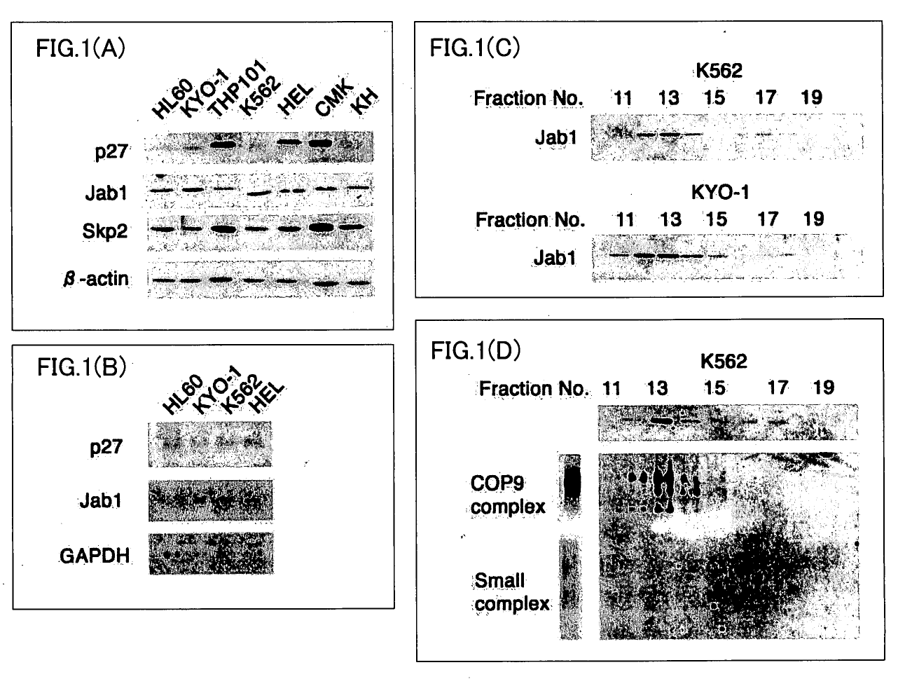

The following will describe the present invention in more detail based on Examples and FIG. 1 through FIG. 7. It should be noted however that the present invention is not limited by the following description. First, cell lines and specific experimental methods used in different Examples will be described.

λExperimental Methodsπ

(1) Leukemia Cell Lines Used

HL60: human acute promyelocytic leukemia cell line. No expression of Bcr-Abl protein. Resistant to Bcr-Abl inhibitor.

THP101: human acute monocytic leukemia cell line. No expression of Bcr-Abl protein. Resistant to Bcr-Abl inhibitor.

HEL: human erythrocytic leukemia cell line. No expression of Bcr-Abl protein. Resistant to Bcr-Abl inhibitor.

CMK: human megakaryocytic leukemia cell line. No expression of Bcr-Abl protein. Resistant to Bcr-Abl inhibitor.

KH: human myelocytic cell line. No expression of Bcr-Abl protein. Resistant to Bcr-Abl inhibitor.

K562: human CML cell line. Expression of Bcr-Abl protein. Sensitive to Bcr-Abl inhibitor.

MEG: human CML cell line. Expression of Bcr-Abl protein. Sensitive to Bcr-Abl inhibitor.

TS9.22: human CML cell line. Expression of Bcr-Abl protein. Sensitive to Bcr-Abl inhibitor.

MOLM1: human CML cell line. Expression of Bcr-Abl protein. Sensitive to Bcr-Abl inhibitor.

KYO-1: human CML cell line. Expression of Bcr-Abl protein. Resistant to Bcr-Abl inhibitor. See Leukemia Research 9: 921-926, (1985).

BAF3: Mouse pro-B cell line.

TonB: Cell line derived from BAF3 and capable of inducing expression of Bcr-Abl gene upon treatment with tetracycline or its analog Doxycyclin (Dox.). See Klucher K M, Lopez D V, Daley G Q. Secondary mutation maintains the transformed state in BaF3 cells with inducible BCR/ABL expression. Blood 91:3927-3934, 1998.

(2) Inhibitors used in the experiments

MG132: proteasome inhibitor

LLM: calpain inhibitor

LMB: nuclear export inhibitor

Cur: CSN satellite protein kinase inhibitor

H89: A kinase inhibitor

Stau. protein kinase inhibitor

PD: MAP kinase inhibitor

Wort.: PI3 kinase inhibitor

(3) Experimental methods

[Preparation of Cell Extract]

Cells in a test sample were collected by centrifugation and washed with a saline solution. The cells were then suspended and dissolved well in a minimum amount of extract buffer solution (50 mM Tris, pH 8.0; 120 mM NaCl, 1 mM EDTA, 0.1% Digitonin). Then, the insoluble component was removed by centrifugation, and the soluble component in the supernatant was extracted as a cell extract.

[Native-PAGE, SDS-PAGE, Western Blotting, Northern Blotting]

These were performed by ordinary methods.

[Fractionation by Glycerol Linear Density Gradient]

One ml of cell extract was fed to 10% to 40% glycerol gradient containing 10 mM Tris-HCl (pH 8.0), 120 mM NaCl, 1 mM EDTA, and 1 mM β-ME. After 24-hour centrifugation (Beckman, using a Ti40 rotor) performed at 4° C. and 27,000 rpm, a 0.5 ml fraction was collected from the bottom.

[Treatment with Tyrosine Kinase Inhibitor]

One to two μM STI571 was directly added to a cell culture.

### Example 1

### Analysis of the Relationship Between p27 Protein Amount and Jab1 Complex in Leukemia Cell Lines

The following cell lines were used as the leukemia cell lines: HL60, KYO-1, THP101, K562, HEL, Mak3, CMK, and KH. From each cell line, a cell extract was prepared, and the expression levels of p27 protein, Jab1 protein, Skp2 protein, and β-actin were detected by Western blotting. The results are shown in FIG. 1(A). Note that, the Skp2 protein, β-actin, and GAPDH protein are indices indicating that the amounts of samples were equivalent.

Next, RNA was extracted from the cells of the leukemia cell lines HL60, KYO-1, K562, and HEL, and the expression level of mRNA in the p27 protein, Jab1 protein, and GAPDH protein was detected by Northern blotting. The results are shown in FIG. 1(B). Note that, the GAPDH protein is an index indicating that the amounts of samples were equivalent.

Thereafter, cell extracts were prepared from the cells of the leukemia cell lines K562 and KYO-1, and the cell extracts were fractionated using a 10% to 40% glycerol linear density gradient method. SDS-PAGE was performed for Jab1 protein contained in each of fraction Nos. 11, 13, 15, 17, and 19, and detection was made by Western blotting. The results are shown in FIG. 1(C).

Next, a cell extract was prepared from K562. After performing Native-PAGE, a Jab1 complex was detected with the anti-Jab1 antibody. The results are shown in the left-hand panel in FIG. 1(D). Further, as above, SDS-PAGE was performed for each of fraction Nos. 11, 13, 15, 17, and 19 of the cell extracts fractionated by a 10% to 40% glycerol linear density gradient method. The results are shown in the upper right panel in FIG. 1(D). Further, these fractions were subjected to Native-PAGE, and a Jab1 complex was detected by a Western blotting method using the anti-Jab1 antibody. The results are shown in the lower right panel in FIG. 1(D). In FIG. 1(D), the notations “COP9 complex” and “Small complex” indicate Jab1 large complex and Jab1 small complex of the Jab1 complex, respectively.

It is clear from the results shown in FIG. 1(A) through FIG. 1(D) that the expression level of p27 protein greatly differs between the different leukemia cell lines, and that the difference is not dependent on mRNA, i.e., there is no control on the transcription level. This suggests the presence of some control on the expression level over the amount of p27 protein.

### Example 2

### Comparison of Jab1 Small Complex Amounts Between Leukemia Cell Lines

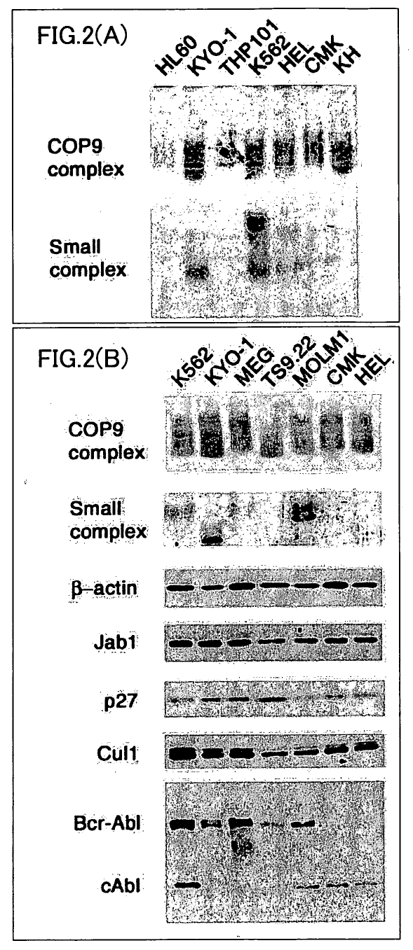

Native-PAGE was performed for each cell extract of the leukemia cell lines prepared in Example 1. This was followed by Western blotting using the anti-Jab1 antibody. The results are shown in FIG. 2(A). In FIG. 2(A), the notations “COP9 complex” and “Small complex” indicate Jab1 large complex and Jab1 small complex of the Jab1 complex, respectively.

Next, the leukemia cell lines K562, KYO-1, MEG, TS9.22, and MOLM1 were selected as CML cell lines, and CMK and HEL were selected as non-CML cell lines. A cell extract was prepared from each of these cell lines. For each cell extract, Native-PAGE was performed, followed by Western blotting using the anti-Jab1 antibody. The results are shown in the first and second panels in the FIG. 2(B).

Further, these cell extracts were subjected to SDS-PAGE to determine expression levels of β-actin, Jab1 protein, p27 protein, Cul1 protein, Bcr-Abl protein (P210 protein), and cAbl protein. The results were analyzed by Western blotting. The results are shown in the third through seventh panels of FIG. 2(B).

It is clear from the results shown in FIG. 2(A) and FIG. 2(B) that the expression level of Jab1 small complex was greater in the CML cell lines than in the non-CML cell lines. It also became clear that the Jab1 small complex exists in at least two different forms.

Sensitivity of each cell line to the tyrosine kinase inhibitor was also examined. The result showed that K562, MEG, MOLM1 were sensitive to the tyrosine kinase inhibitor, and that KYO-1, CMK, and HEL were resistant to the tyrosine kinase inhibitor. By comparing these results with the different forms of Jab1 small complex, it was found that only the cells with the K562-type Jab1 small complex were sensitive to the tyrosine kinase inhibitor.

### Example 3

### Response of K562 Line and KYO-1 Line of CML Cell Lines to Tyrosine Kinase Inhibitor

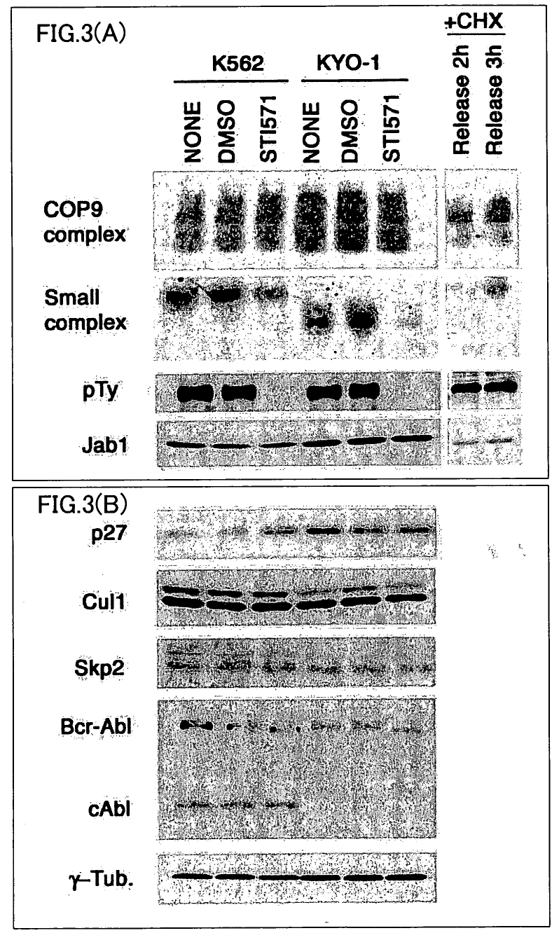

The Bcr-Abl tyrosine kinase sensitive line K562 and the Bcr-Abl tyrosine kinase resistant line KYO-1 were selected from the CML cell lines. The selected cell lines were then treated with DMSO or ST571 (Bcr-Abl tyrosine kinase specific inhibitor) for 8 hours, and a cell solution was prepared from each cell line. The cell extract was then subjected to Native-PAGE, and changes in the Jab1 complex were analyzed by Western blotting using the anti-Jab1 antibody. The results are shown in the first and second panels on the left-hand side of FIG. 3(A).

These cell extracts were also subjected to SDS-PAGE, and the level of phosphorylated tyrosine was analyzed by Western blotting. The results are shown in the third panel on the left-hand side of FIG. 3(A). In the same manner, the cell extracts were subjected to SDS-PAGE, and the expression level of Jab1 protein was analyzed by Western blotting. The results are shown in the fourth panel on the left-hand side of FIG. 3(A).

Further, K562 was treated for 8 hours with ST571. After the treatment, ST571 was removed and incubated for 2 hours and 3 hours in the presence of cycloheximide (CHX), which is a protein synthesis inhibitor. Then, analysis was made on changes in the Jab1 complex, the level of phosphorylated tyrosine, and the expression level of Jab1 protein. The results are shown in the right-hand panels of FIG. 3(A). In FIG. 3(A), the first and second panels on the right-hand side show changes in the Jab1 complex, the third panel indicates the level of phosphorylated tyrosine, and the fourth panel on the right-hand side indicates the expression level of Jab1 protein.

Further, the cell extracts were subjected to SDS-PAGE, and the expression levels of p27 protein, Cul1 protein, Skp2 protein, Bcr-Abl protein, cAbl protein, and γ-Tub protein were determined by Western blotting. The results are shown in FIG. 3(B). Note that, the γ-Tub protein is an index indicating that the amounts of samples were equivalent.

It is clear from the results shown in FIG. 3 that inhibiting the tyrosine kinase activity of the product of the Bcr-Abl gene reduces the amount of Jab1 small complex in both K562 line and KYO-1 line. As to the p27 protein, which is a negative marker for malignant cancer, there was an increase only in the K562 line. That is, cancer malignancy was reduced and the efficacy of the tyrosine kinase inhibitor was improved only in the K562 line sensitive to the tyrosine kinase inhibitor.

It became clear from these results that forms of existence of the Jab1 small complex correspond to the efficacy of the tyrosine kinase inhibitor. Thus, the efficacy of the tyrosine kinase inhibitor can be determined if the forms of existence (K562 line type or KYO-1 line type) of the Jab1 small complex derived from cancer cells were checked prior to administration of the tyrosine kinase inhibitor or other anticancer drugs.

### Example 4

### Relationship Between Bcr-Abl Protein and Jab1 Complex

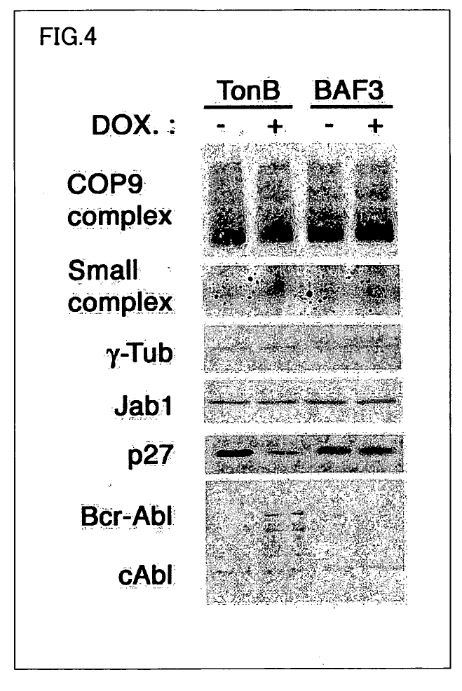

Using BAF3 and TonB, these cells were treated for 5 hours in the absence of IL3 and in the presence of Dox., or in the absence of Dox. These cell lines were then collected, and a cell extract was prepared from each cell line. Then, analysis was made on changes in the Jab1 complex, and the expression level of γ-Tub, Jab1 protein, p27 protein, Bcr-Abl protein, and cAbl protein, using Western blotting. The results are shown in FIG. 4.

As can be seen from the results shown in FIG. 4, the amount of Jab1 small complex was increased by the induced expression of Bcr-Abl protein.

### Example 5

### Analysis of Signal Transduction Pathways Controlling the Jab1 Small Complex

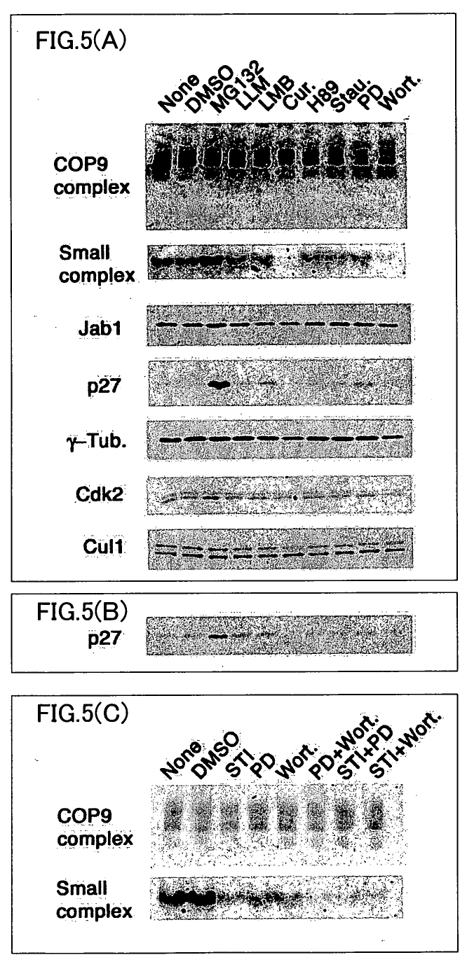

K562 cells were treated for 8 hours with MG132, LLM, LMB, Cur., H89, Stau., PD, and Wort., and a cell extract was prepared for each sample. With the cell extracts, analysis was made on changes in the Jab1 complex, and the expression level of Jab1 protein, p27 protein, γ-Tub protein, Cdk2 protein, and Cul protein, using Western blotting. The results are shown in FIG. 5(A). Note that, the Cdk2 protein and Cul protein are indices indicating that the amounts of samples were equivalent.

The same process was performed with KYO-1, and the expression level of p27 protein was analyzed by Western blotting. The results are shown in FIG. 5(B).

Further, K562 was treated for 8 hours with STI alone, PD alone, and Wort. alone. The treatment was also performed for 8 hours with a combination of PD and Wort., a combination of STI and PD, and a combination of STI and Wort. Then, changes in the Jab1 complex were analyzed by Western blotting. The results are shown in FIG. 5(C).

As is clear from the results shown in FIG. 5, the Jab1 small complex decreased as a result of inhibiting MAPK pathway and PI3K pathway.

As can be seen from the results of Examples 1 through 5, the Bcr-Abl tyrosine kinase constantly activates MAPK pathway and PI3K pathway in CML. As a result, the amount of Jab1 small complex increases in the cell. Further, there was a negative correlation between the amount of Jab1 small complex and the expression level of p27 protein. The inventors of the present invention have reported that overexpression of Jab1 protein increases the amount of Jab1 small complex and induces degradation of p27 protein, and that the degradation of p27 protein by the Jab1 small complex is inhibited by the nuclear export inhibitor leptomycin B (LMB). (See Tomoda K et al. J Biol Chem. 2002 Jan. 18; 277(3): 2302-10, and the program and lecture summary for the 25th Annual Meeting of the Molecular Biology Society of Japan, 2P-0760, organizing committee of the 25th Annual Meeting of the Molecular Biology Society of Japan, published on Nov. 25, 2002.) By considering these results together, there is a possibility that the increased amount of Jab1 protein facilitates degradation of p27 protein. It is therefore considered that the control of Jab1 complex by Bcr-Abl protein is one factor of producing cancer cells. A method of determining the efficacy of anticancer drugs according to the present invention is realized based on these findings.

The embodiments and concrete examples of implementation discussed in the foregoing detailed explanation serve solely to illustrate the technical details of the present invention, which should not be narrowly interpreted within the limits of such embodiments and concrete examples, but rather may be applied in many variations within the spirit of the present invention, provided such variations do not exceed the scope of the patent claims set forth below.

### INDUSTRIAL APPLICABILITY

As described above, the present invention uses Jab1 protein as an index, and checks the forms of existence of the Jab1 protein to determine the efficacy of anticancer drugs. Thus, with the present invention, the effectiveness of the anticancer drug can be improved, and the cancer patients do not need to endure the side effects or huge cost of unnecessarily administered anticancer drugs.

The present invention is therefore suitable for a wide variety of industries, including laboratory testing, pharmaceutical industry, and research reagent industry. The invention is also applicable in the field of medicine.

1. A method of determining the efficacy of an anticancer drug used for cancer treatment, wherein Jab1 protein contained in a test sample is used as an index of determination.

2. A method as set forth in claim 1, comprising:
an extract preparing step of preparing an extract from a test sample containing cancer cells; and,
a Jab1 checking step of checking for a form of existence of Jab1 protein contained in the extract.

3. A method as set forth in claim 2, further comprising a fractionating step of fractionating the extract based on molecular weight, wherein the extract fractionated in the fractionating step is used in the Jab1 checking step.

4. A method as set forth in claim 2, wherein, in the Jab1 checking step, a molecular weight of a Jab1 complex formed by the Jab1 protein is used as a reference to check for a form of existence of Jab1 protein.

5. A method as set forth in claim 4, wherein the Jab1 complex exists as a Jab1 large complex with a molecular weight of 450 kDa, and/or a Jab1 small complex with a molecular weight of 120 kDa, and wherein, in the Jab1 checking step, an expression level of the Jab1 small complex is checked to check for a form of existence of Jab1 protein.

6. A method as set forth in claim 4, comprising a p27 checking step of checking for expression of p27 protein contained in the test sample.

7. A method as set forth in claim 4, wherein, in at least one of the Jab1 checking step and the p27 checking step, an expression level of the Jab1 small complex or protein is checked by electrophoresis.

8. A method as set forth in claim 7, wherein, in the Jab1 checking step, the electrophoresis is performed by native-polyacrylamide gel electrophoresis.

9. A method as set forth in claim 4, wherein, in the Jab1 checking step, the Jab1 small complex is detected from the extract using an antibody that specifically binds to the Jab1 protein.

10. A method as set forth in claim 4, wherein, in the p27 checking step, the p27 protein is detected from the extract using an antibody that specifically binds to the p27 protein.

11. A method as set forth in claim 9, wherein the antibody comprises one of a monoclonal antibody and a polyclonal antibody.

12. A method as set forth in claim 1, wherein the cancer is leukemia.

13. A method as set forth in claim 12, wherein the leukemia is Chronic Myelogenous Leukemia.

14. A method as set forth in claim 1, wherein the anticancer drug is a tyrosine kinase inhibitor.

15. A method as set forth in claim 1, which is performed prior to administration of the anticancer drug.

16. An anticancer drug efficacy determination kit for performing a method of claim 1.

17. An anticancer drug efficacy determination kit which comprises a group of reagents for checking an expression state of Jab1 protein contained in an extract prepared from a test sample including cancer cells.

18. An anticancer drug efficacy determination kit as set forth in claim 17, which comprises an antibody that specifically binds to Jab1 protein.

19. An anticancer drug efficacy determination kit as set forth in claim 17, wherein the Jab1 protein exists as a Jab1 large complex with a molecular weight of 450 kDa, and/or a Jab1 small complex with a molecular weight of 120 kDa, and wherein the group of reagents contains at least the Jab1 small complex as a marker for polyacrylamide gel electrophoresis.
